(19) United States
(12) Patent Application Publication
(10) Pub. No.: US 2025/0191800 A1
(43) Pub. Date: Jun. 12, 2025

(54) METHOD AND SYSTEM FOR THE CONCENTRATION OF RADIUM AND ACTINIUM FROM DILUTE SOLUTIONS

(71) Applicant: TerraPower, LLC, Bellevue, WA (US)

(72) Inventors: Christopher P. Dunckley, Seattle, WA (US); Laura Margaret Lilley, Seattle, WA (US)

(21) Appl. No.: 18/974,264

(22) Filed: Dec. 9, 2024

Related U.S. Application Data
(60) Provisional application No. 63/607,357, filed on Dec. 7, 2023.

(51) Int. Cl. G21G 7/00; A61K 51/12; B01D 15/18; B01D 15/36; B01D 15/42; B01J 20/22

(52) U.S. Cl. CPC G21G 1/001; A61K 51/121; B01D 15/1871; B01D 15/362; B01D 15/426; B01J 20/26; G21G 2001/0089

(57) ABSTRACT

Disclosed herein are methods and systems for the use of extraction chromatography resins to concentrate Ac from dilute solutions that may contain Th isotopes and/or other isotopes, such as Ra. As disclosed herein, at least some extraction chromatography resins strongly retain trivalent actinides between 4 and 6 M HNO₃ that can be recovered in good yields with small volumes of dilute acids. These small volumes of acid eluents result in significant concentration of the solution of the trivalent/certain tetravalent ions, such that they can be further processed without requiring lengthy evaporation steps that are utilized in standard Ac concentration methods.

### CROSS-REFERENCE TO RELATED APPLICATIONS

This application claims the benefit of U.S. Provisional Patent Application No. 63/607,357, filed Dec. 7, 2023, which is incorporated by reference herein in its entirety.

### INTRODUCTION

Standard methods in the literature rely on evaporation to concentrate dilute trivalent metals including rare earth metals and actinides. Particularly, common radiochemical processing schemes for isolating the medical radioisotope actinium (Ac), particularly actinium-225 (Ac-225), use multiple large volume evaporation steps. For example, thorium-229 (Th-229) undergoes alpha decay to form Ra-225, which undergoes beta decay to form Ac-225. Standard methods separate Th-229 from radium-225 (Ra-225) and/or Ac-225 using an anion exchange resin (for example, a resin such as AG MP-1M or TEVA) followed by an evaporation, prior to separation of the desired Ac-225 from the Ra-225 using DGA resin (that utilizes, for example, tetraoctyl diglycolamide). While there are methods that concentrate the Ac-225 from bulk solutions relying on cation resins, such methods require harsh chemical conditions to recover the Ac-225; for at least this reason, evaporation followed by anion exchange remains the favored standard technique. Other known methods have similar drawbacks when compared to evaporation. Stable metal precipitation (for example, utilizing stable metals such as La) requires further separations to remove the bulk metal used to co-precipitate the Ac-225. There are numerous methods reported for the concentration of Ra isotopes (for example, Ra-228 and Ra-226) from environmental samples (e.g., sea water/tap water), however, these methods do not account for the high concentrations of acid(s) used in Ac-225 processing.

### METHOD AND SYSTEM FOR THE CONCENTRATION OF RADIUM AND ACTINIUM FROM DILUTE SOLUTIONS

A novel approach is proposed herein for the use of extraction chromatography resins to concentrate Ac from dilute solutions that may contain Th isotopes and/or other isotopes, such as Ra. As disclosed herein, at least some extraction chromatography resins strongly retain trivalent actinides between 4 and 6 M HNO₃ that can be recovered in good yields with small volumes of dilute acids (e.g., HNO₃ or HCl). These small volumes of acid eluents result in significant concentration of the solution of the trivalent/certain tetravalent ions, such that they can be further processed without requiring lengthy evaporation steps that are utilized in standard Ac concentration methods.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended as a definition of the limits of a particular example. The figures, together with the remainder of the specification, serve to explain principles and operations of the described and claimed aspects and examples. For purposes of clarity, not every component may be labeled in every figure.

FIG. 1 illustrates an example process for concentrating Ac from a dilute stream.

FIG. 2 illustrates an example system for concentrating Ac and/or other actinides or lanthanides from a dilute stream.

FIG. 3 illustrates an example method for concentrating Ac from a dilute stream.

FIG. 4 illustrates an example method for concentrating Ra from a dilute stream.

FIG. 5A illustrates experimental data showing a mass of La recovered during a process utilizing an extraction chromatography resin.

FIG. 5B illustrates experimental data showing a mass percentage of La recovered during a process utilizing an extraction chromatography resin.

FIG. 6A illustrates experimental data showing a mass of La recovered during a process utilizing an extraction chromatography resin.

FIG. 6B illustrates experimental data showing a mass percentage of La recovered during a process utilizing an extraction chromatography resin.

FIG. 7A illustrates experimental data showing a mass of La recovered during a process utilizing an extraction chromatography resin.

FIG. 7B illustrates experimental data showing a mass of Th recovered during a process utilizing an extraction chromatography resin.

FIG. 8A illustrates experimental data showing a mass percentage of La recovered during a process utilizing an extraction chromatography resin.

FIG. 8B illustrates experimental data showing a mass percentage of Th recovered during a process utilizing an extraction chromatography resin.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Examples may be practiced as methods, systems, or devices. It should be understood that this invention is not limited to the particular methodology, protocols, and reagents, etc., described herein and as such may vary. The following detailed description is therefore not to be taken in a limiting sense, and the scope of the present disclosure is defined by the appended claims and their equivalents.

Unless defined otherwise, all technical and scientific terms used herein have the same meaning as those commonly understood to one of ordinary skill in the art to which this invention pertains.

For the purposes of this application the following terms shall have the following meanings:

As used herein and in the claims, the singular forms “a,” “an”, and “the” include the plural reference unless the context clearly indicates otherwise.

Other than in the operating examples, or where otherwise indicated, all numbers expressing quantities of ingredients or reaction conditions used herein should be understood as modified in all instances by the term “about.” The term “about” when used herein in connection with numerical values means±20% and with percentages means±5%.

As used herein, the term “comprising” refers to a composition, compound, formulation, or method that is inclusive and does not exclude additional elements or method steps.

As used herein, the term “consisting of” refers to a compound, composition, formulation, or method that excludes the presence of any additional component or method steps.

Before the methods and systems are disclosed and described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments of the methods and systems disclosed and is not intended to be limiting. It must be noted that, as used in this specification, the singular forms “a,” “an,” and “the” include plural referents unless the context clearly dictates otherwise. Thus, for example, reference to “a thorium isotope” is not to be taken as quantitatively or source limiting, reference to “a step” or “an operation” may include multiple steps or operations, reference to “producing” or “products” of a reaction should not be taken to be all of the products of a reaction, and reference to “reacting” may include reference to one or more of such reaction steps. As such, the step of reacting can include multiple or repeated reaction of similar materials to produce identified reaction products.

Disclosed herein are methods and systems for the use of extraction chromatography resins to concentrate Ac (for example, Ac-225) directly from dilute (for example, about 10 ppm Ac or less) solutions that may contain Th isotopes and/or other isotopes, such as Ra. In a finding opposite from that expected from an inspection of the current standard methods for processing of dilute solutions, experiments described herein determined that at least some extraction chromatography resins strongly retain trivalent actinides between 4 and 6 M HNO₃ that can be recovered in good yields with small volumes of dilute acids (e.g., HNO₃ or HCl). These small volumes of acid eluents result in significant concentration of the solution of the trivalent/certain tetravalent ions, such that they can be further processed without requiring lengthy evaporation steps. Further, the methods and systems also allow use of downstream exchange resins such as cation exchange, Sr/Pb/Ln resins, or other resins to concentrate Ra (for examples, Ra-225) from dilute solutions.

As noted above, standard methods of concentrating Ac include concentrating a dilute stream of Ac and Ra via an evaporation process, and then passing that concentrated Ac and Ra stream through a resin column to separate the Ac from any Th and/or Ra in the stream. An example of such a process is described in U.S. Published Application No. 2022/0339292, titled Titania Based Generators for Ac-225 Generation (the '292 Application), is hereby incorporated herein by reference in its entirety. The '292 Application describes a system in which thorium (Th) is retained on a resin in a generator column and from which a dilute stream of Ac and Ra is periodically washed. The dilute stream is then concentrated by evaporation and the concentrated Ra and Ac stream is subsequently passed through a UTEVA (Uranium and Tetravalent Actinides) resin column to separate the Ra and Ac (as well as any Th that may have escaped the generator column).

The improved methods and systems disclosed herein replace the evaporation step(s) utilized in standard methods with an extraction chromatography resin step as described herein. In some examples, instead of the concentrated (post-evaporation) solution of Ac and Ra being transferred to a UTEVA resin at a downstream column, only a concentrated Ac output from the new extraction chromatography resin column (which may also be referred to as an Ac concentration column) may be passed to a downstream column (for example, one containing a UTEVA resin).

As discussed herein and as shown by the examples, herein, extraction chromatography resins (for example, BDGA resin) captures Ac at dilute concentrations (in some examples, at concentrations of between about 3 M HNO₃ and about 7 M HNO₃ (in further examples, at concentrations of about 5 M HNO₃)). Further, it is shown herein that, surprisingly, the Ac is captured at high yields in the disclosed processes despite a large ratio of (load solution volume to bed volume); the Ac is then eluted in a small number of bed-volumes, such that there is an overall concentration effect over the column of greater than 30×. The combination of low concentration (for example, of Ac and/or Ra) and high volume through the extraction chromatography column unexpectedly are shown to efficiently result in concentration of Ac; this is a marked improvement over the current/standard systems and methods that cannot achieve such separation with such large volumes and such dilute streams.

Elimination of the evaporation step provides significant improvements over standard methods. One benefit of the disclosed methods and processes is a reduction in cost and complexity, as evaporation processes of radioactive materials is often complex and expensive. Another benefit of the disclosed methods and systems is that the extraction chromatography resin will produce an Ac output containing less Th than the standard evaporating methods, therefore less Th will reach a downstream column or other downstream process (for example, a UTEVA resin column).

An additional benefit to the disclosed methods and systems is that Ra that may be included with the Ac in the dilute stream is separated from the desired product (the Ac) earlier in the process (e.g. at the extraction chromatography resin step instead of at the downstream column/UTEVA resin step). This reduces the activity of the streams downstream of the extraction chromatography resin step and simplifies handling the of concentrated Ac stream after separation. Further, the dilute Ra output stream from the new extraction chromatography step may be treated separately, for example, by yet another extraction chromatography resin column or other type of column or separation that is provided specifically to concentrate the Ra from the dilute Ra stream. Such concentration of the Ra stream allows for both capture of the Ra in its own right, and further ingrowth of any residual Ac that can be subsequently separated from the dilute Ra stream and added to the concentrated Ac stream.

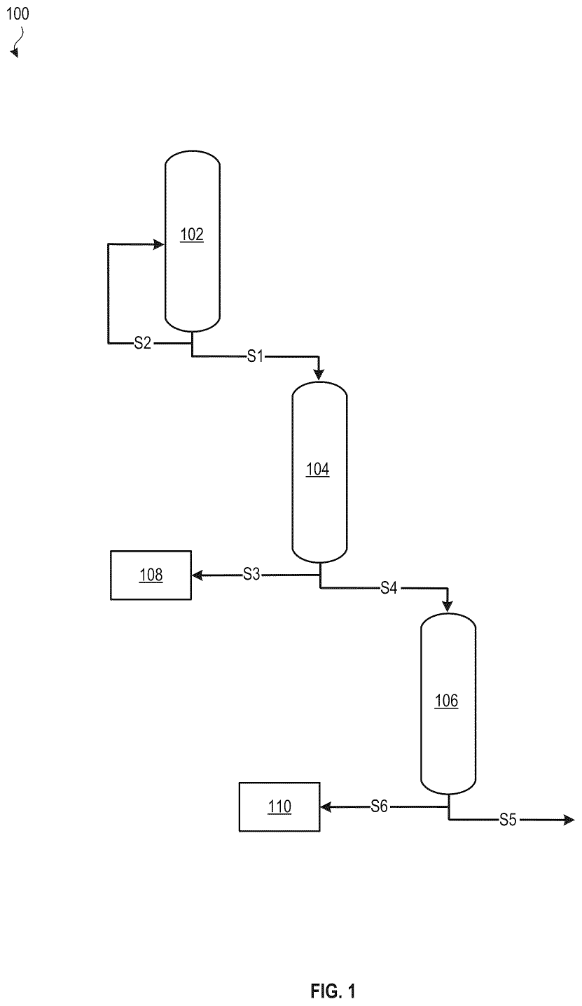

FIG. 1 illustrates an example process 100 for concentrating Ac from a dilute stream. A generator operation 102 that includes Th may generate, as an output, a dilute stream S1 that contains Ac (and, in some examples, Ra). In examples, the Th is Th-229, the Ra is Ra-225, and the Ac is Ac-225. In some examples, generator column may include a resin such as AG MP-1M (for example, which may be obtained from BioRad Laboratories, Inc., 1000 Alfred Nobel Drive, Hercules, California 94547). In examples, within the generator operation 102, Th-229 will subsequently decay over time to generate its daughter products, including Ra-225 and Ac-225.

In examples, dilute stream S1 contains an acid, for example, HNO3, HCl, NaNO3, HOA (acetic acid), and/or NaOA (sodium acetate), or others). In some examples, reconstituted Th that escapes the generator operation 102 may be captured and returned as stream S2 to the generator operation 102.

Dilute stream S1 is received at an inlet of an extraction chromatography column 104. Extraction chromatography column 104 may have a body defining an interior chamber that contains an extraction chromatography resin. In some examples, the extraction chromatography resin is N,N,N′,N′-tetra-n-octyldiglycolamide (DGA Resin, Normal) and/or N,N,N′,N′-tetra-2-ethylhexyldiglycolamide (DGA Resin, Branched, also referred to as BDGA) resins, available from Eichrom Technologies, Inc., 1955 University Lane, Lisle, IL, 60532. At the extraction chromatography column 104, Ac is retained within the interior chamber (e.g. is loaded onto the extraction chromatography resin) and a dilute Ra stream S3 is removed from the extraction chromatography column 104. In examples, the extraction chromatography resin may include one or more other appropriate resins with affinity for Ac.

Dilute Ra stream S3 may include Ra, some Ac, some Th, and/or other components. Dilute Ra stream S3 may be processed (for example, concentrated and/or separated) at a downstream Ra-processing operation 108. In some examples, Ra-processing operation 108 may separate Ra from other components of the dilute Ra stream S3 that form the waste stream of the Ra-separation process, although the other components may be further utilized. For example, Ra-processing operation 108 may separate the Ra from Ac, the latter of which may be recovered and combined with Ac generated by extraction chromatography column 104 or a further downstream Ac processing column 106. In examples, Ra-processing operation 108 may separate the Ra from Th, the latter of which may be recovered and reconstituted for feeding back into the generator operation 102. In some examples, Ra-processing operation 108 may include a column having a column body defining an interior chamber that contains an Ra-retaining resin. In some examples, the Ra-retaining resin may include one or more of a Sr resin, a Pb resin, an Ln resin (for example, which may be obtained from Eichrom Technologies, Inc., 1955 University Lane, Lisle, IL, 60532), a cation resin (for example, AmberChrom (formerly Dowex) 50WX8 cation exchange resin, which may be obtained from DuPont, headquartered at 974 Centre Rd. Bldg. 730, Wilmington, DE, 19805; or AG 50W-X12, which may be obtained from BioRad Laboratories, Inc., 1000 Alfred Nobel Drive, Hercules, California 94547), or another suitable resin type. In some examples, the Ra retained on the Ra-retaining resin may be eluted from the resin by a wash liquid, for example, a wash fluid containing an acid, for example, HNO₃, HCl, NaNO₃, HOA (acetic acid), and/or NaOA (sodium acetate), or others).

In examples, the retained Ac is eluted from the extraction chromatography resin and is removed from the extraction chromatography column 104 as an Ac stream or eluent/eluted stream S4. In some examples, elution is performed with a wash fluid containing an acid or other compound, for example, H₂O₂, HNO₃, HCl, NaNO₃, HOA (acetic acid), and/or NaOA (sodium acetate), or others).

In some examples the Ac stream S4 is received at a downstream Ac processing column 106. Column 106 may include a column body that defines an interior chamber that contains an Ac-retaining resin. In some examples, the Ac-retaining resin may include a UTEVA resin, which may be obtained from Eichrom Technologies, Inc., 1955 University Lane, Lisle, IL, 60532, or another suitable resin type.

In some examples, an outlet stream S5 may include residual Ra, Th, and/or other compounds of the Ac stream S4, which may be further separated and/or disposed, in some examples. In examples, the retained Ac is eluted from the Ac-retaining resin of column 106 and is removed from the column 106 as a concentrated Ac stream or eluent/eluted stream S6. In some examples, elution is performed with a wash fluid containing an acid or other compound, for example, H₂O₂, HNO₃, HCl, NaNO₃, HOA (acetic acid), and/or NaOA (sodium acetate), or others). In some examples, the concentrated Ac stream S6 may be further concentrated, separated, and/or otherwise processed or utilized at downstream operations 110.

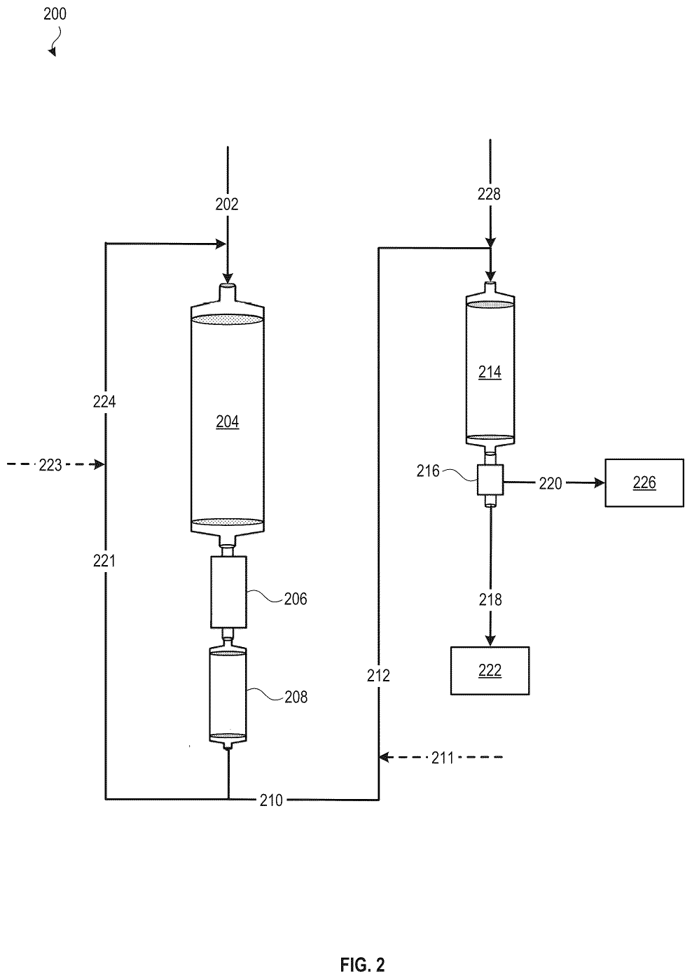

FIG. 2 illustrates an example system 200 for concentrating Ac and/or other actinides or lanthanides from a dilute stream. In examples, system 200 may represent a portion of a process such as process 100. A starting composition 202 enters a generator column 204 (for example, such as generator operation 102).

An outlet stream of the generator column 204 may pass through a selectable valve 206 and/or a filter 208. Selectable valve 206 may be able to be placed in at least two positions. In an example position, a stream 221 of Th that escapes from the generator column 204 may be reconstituted and returned to the inlet 202 of the generator column 204. In a particular example, the stream 221 may exit the filter 208 at a Th concentration of about 0.05 M HNO₃, and may be reconstituted to a concentrated stream 224 of about Th 8 M HNO₃ (for example, by addition of stream 223), before entering the column again as part of a starting composition 202.

In another example position of selectable valve 206, an effluent (outlet) stream 210 containing Ac and Ra (and, in some examples, escaped Th), may be routed to (for example, passed, flowed, injected, or otherwise delivered into) an extraction chromatography column 214 (for example, such as extraction chromatography column 104). In a particular example, the concentration of effluent stream 210 may be about Ra/Ac 8 M HNO₃. In some examples, the concentration of dilute stream 210 may be between about Ra/Ac 3 M HNO₃ and about Ra/Ac 13 M HNO₃. In some examples, the concentration of dilute stream 210 may be between about Ra/Ac 5 M HNO₃ and about Ra/Ac 10 M HNO₃. In some examples, the concentration of dilute stream 210 may be between about Ra/Ac 3 M HNO₃ and about Ra/Ac 8 M HNO₃. In some examples, the concentration of dilute stream 210 may be between about Ra/Ac 8 M HNO₃ and about Ra/Ac 13 M HNO₃.

In some examples, the effluent stream 210 may be diluted to form diluted effluent stream 212, for example, by addition of one or more acids 211 prior to the extraction chromatography column 214. In such examples, the diluted effluent stream 212 is then routed to and passed into (for example, flowed, injected, or otherwise delivered into) the extraction chromatography column 214. In a particular example, the concentration of dilute effluent stream 212 may be about Ra/Ac 5 M HNO₃. In some examples, the concentration of dilute effluent stream 212 may be between about Ra/Ac 0.5 M HNO₃ and about Ra/Ac 10 M HNO₃. In some examples, the concentration of dilute effluent stream 212 may be between about Ra/Ac 2 M HNO₃ and about Ra/Ac 8 M HNO₃. In some examples, the concentration of dilute effluent stream 212 may be between about Ra/Ac 0.5 M HNO₃ and about Ra/Ac 5 M HNO₃. In some examples, the concentration of dilute effluent stream 212 may be between about Ra/Ac 5 M HNO₃ and about Ra/Ac 10 M HNO₃.

At the extraction chromatography column 214, which contains an extraction chromatography resin, Ac is bound onto the extraction chromatography resin. In some examples, a column loading flow rate is about 9 mL/min. In some examples, a column loading flow rate is between about 5 mL/min and about 15 mL/min. In some examples, a column loading flow rate is between about 2 mL/min and about 18 mL/min. In some examples, a column loading flow rate is between about 2 mL/min and about 9 mL/min. In some examples, a column loading flow rate is between about 9 mL/min and about 18 mL/min.

An outlet stream of the extraction chromatography column 214 may pass through a selectable valve 216. Selectable valve 216 may be able to be placed in at least two positions. In an example position, for example, during loading of the extraction chromatography column 214, the selectable valve 216 may direct a dilute Ra outlet stream 218 that is removed from the extraction chromatography column 214 to an Ra-processing operation 222 (for example, at one or more Ra-processing operations such as Ra-processing operations 108). In a particular example, the concentration of dilute Ra outlet stream 218 may be about Ra 5 M HNO₃. In some examples, the concentration of dilute Ra outlet stream 218 may be between about Ra 0.5 M HNO₃ and about Ra 10 M HNO₃. In some examples, the concentration of dilute Ra outlet stream 218 may be between about Ra 2 M HNO₃ and about Ra 8 M HNO₃. In some examples, the concentration of dilute Ra outlet stream 218 may be between about Ra 0.5 M HNO₃ and about Ra 5 M HNO₃. In some examples, the concentration of dilute Ra outlet stream 218 may be between about Ra 5 M HNO₃ and about Ra 10 M HNO₃.

In another example position, for example, during washing/eluting of the Ac from the extraction chromatography resin within extraction chromatography column 214, selectable valve 216 may direct an eluted Ac stream 220 that is removed from the extraction chromatography column 214 to one or more Ac-processing operations 226. Elution of the extraction chromatography column 214 may include passing an elution liquid/wash solution 228 through the extraction chromatography column 214. In some examples, an elution speed of the wash liquid/wash solution 228 is about 12 mL/min. In some examples, an elution speed of the wash liquid/wash solution 228 is between about 5 mL/min and about 20 mL/min. In some examples, an elution speed of the wash liquid/wash solution 228 is between about 10 mL/min and about 15 mL/min. In some examples, an elution speed 228 of the wash liquid/wash solution is between about 5 mL/min and about 12 mL/min. In some examples, an elution speed of the wash liquid/wash solution 228 is between about 12 mL/min and about 20 mL/min.

In some examples, the wash liquid/wash solution 228 includes an acid. In some examples, the wash liquid/wash solution 228 includes a concentration of HNO₃ of about 0.01 M to about 10 M. In some examples, the wash liquid/wash solution 228 includes a concentration of HNO₃ of about 0.1 M to about 1.0 M. In some examples, the wash liquid/wash solution 228 includes a concentration of HNO₃ of about 0.1 M to about 10 M. In some examples, the wash liquid/wash solution 228 includes a concentration of HNO₃ of about 0.01 M to about 1.0 M.

As noted above, the eluted Ac stream 220 may be provided to a downstream Ac processing operation 226 for further processing (for example, an operation such as column 106 and/or operations 110). In a particular example, the concentration of Ac stream 220 may be about Ac 0.05 M HNO₃. In some examples, the concentration of Ac stream 220 may be between about Ac 0.005 M HNO₃ and about Ac 0.1 M HNO₃. In some examples, the concentration of Ac stream 220 may be between about Ac 0.02 M HNO₃ and about Ac 0.08 M HNO₃. In some examples, the concentration of Ac stream 220 may be between about Ac 0.005 M HNO₃ and about Ac 0.05 M HNO₃. In some examples, the concentration of Ac stream 220 may be between about Ac 0.05 M HNO₃ and about Ac 0.1 M HNO₃.

In some examples, the all or parts of the system 200 may be utilized at a commercial scale. In some examples, all or parts of the system 200 may be utilized at a laboratory scale for performing experiments. In some examples, all or parts the system 200 (for example, extraction chromatography column 214) may be utilized to perform experiments utilizing lanthanum (La) in lieu of Ac. La and Ac are in the same chemical group, and have the same number of valence electrons. In many applications, the reactivity of La may mimic the reactivity of Ac, and La may be advantageous for experimental use in situations where the supply of Ac is limited, and because La has a naturally occurring stable isotope (La-139) whereas Ac does not.

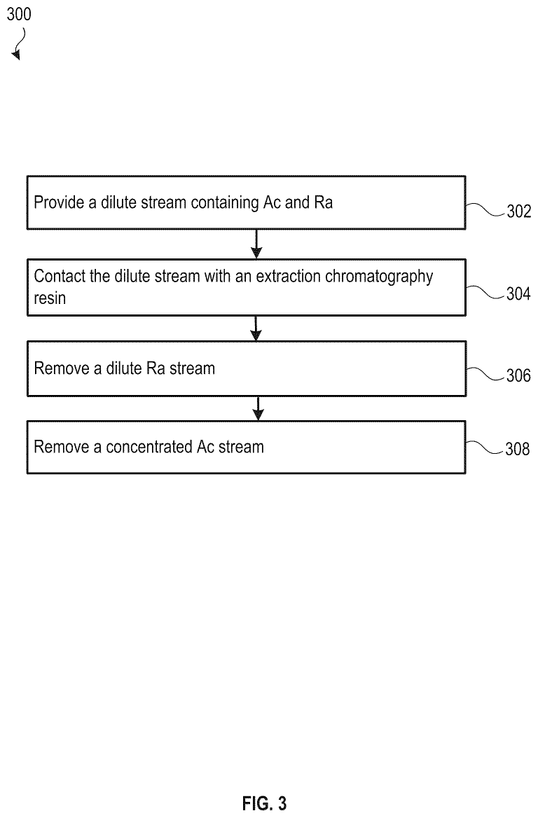

FIG. 3 illustrates an example method 300 for concentrating Ac (for example, Ac-225) from a dilute stream. In some examples, all or parts of method 300 may be carried out as described above regarding FIGS. 1 and 2.

At operation 302, a dilute stream containing Ac and Ra is provided. In some examples, the dilute stream may have originated as an outlet of a generator column containing Th that decays to form its daughter isotopes, such as Ra and Ac.

At operation 304, the dilute stream contacts an extraction chromatography resin, for example, within an extraction chromatography column. In an example, the dilute stream flows into the interior of the column where it contacts the extraction chromatography resin. The dilute stream can flow through or be held within the column for some amount of time before the stream is removed from the column. Within the column, the Ac is bound to/loaded onto the extraction chromatography resin.

At operation 306, a dilute Ra stream is removed (for example, because the Ac is bound to the extraction chromatography resin but the Ra and carrier acid pass through). In some examples, the dilute Ra stream is passed through a second column, containing a second resin, wherein the Ra binds to the second resin.

At operation 308, a concentrated (e.g. eluted) Ac stream is removed. Removal may be done via an elution operation, wherein a washing liquid is passed through the extraction chromatography column to release the Ac from the extraction chromatography resin and wash it out (e.g. removing it from) of the column. This generates an Ac stream (for example, a concentrated Ac stream) that may be further processed for utilization.

In some examples, the Ac (for example, Ac-225) may be further utilized for radiolabeling and/or generation of radiopharmaceuticals, radiotracing, functional testing, medical imaging, and/or others. For example, the Ac may be further purified from the concentrated/eluted Ac stream may be bound to a ligand/linker molecule. Such a ligand may be further bound to a targeting molecule that may target a particular type of cell or tissue.

In some examples, the method 300 and/or its upstream or downstream operations may be performed without an evaporation step. For example, the eluted Ac stream generated at operation 308 may then be utilized in downstream operations (for example, a radiopharmaceutical creation) without the use of an evaporation step. In examples, there may be no evaporation step included between (or upstream of) the operations of method 300, for example, between operations 302 and 304.

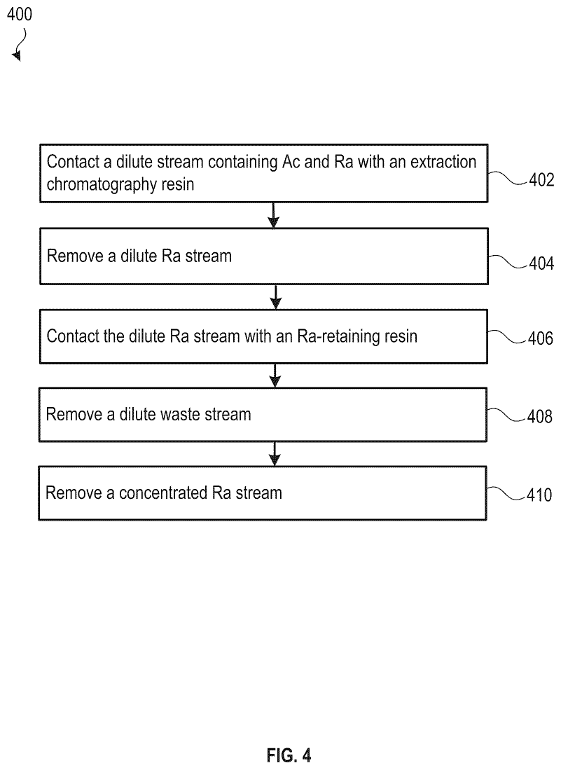

FIG. 4 illustrates an example method 400 for concentrating Ra (for example, Ra-225) from a dilute stream. In some examples, all or parts of method 400 may be carried out as described above regarding FIGS. 1, 2, and 3.

At operation 402, a dilute stream containing Ac and Ra contacts an extraction chromatography resin, for example, within an extraction chromatography column. Within, the Ac is bound to/loaded onto the extraction chromatography resin.

At operation 404, a dilute Ra stream is removed (for example, because the Ac is bound to the extraction chromatography resin but the Ra and carrier acid pass through).

At operation 406, the dilute Ra stream contacts an Ra-retaining resin, for example, at a second column downstream of the extraction chromatography column.

At operation 408, a dilute waste stream is removed from the column (for example, because the Ra is bound to the extraction chromatography resin but other components of the stream pass through). In some examples, the waste stream includes components that may be reconstituted and added to other streams described herein, such as small but valuable amounts of Th and/or Ac.

At operation 410, a concentrated (e.g. eluted) Ra stream is removed. Removal may be done via an elution operation, wherein a washing liquid is passed through the second column to release the Ra from the Ra-retaining resin and wash it out (e.g. removing it from) of the column. This generates an Ra stream (for example, a concentrated Ra stream) that may be further processed for utilization.

### EXAMPLES

### Example 1: La Over BDGA

Example 1 was performed utilizing a laboratory setup as described above with regards to extraction chromatography column 214 of system 200. La was utilized as an analog for Ac. The feed to the extraction chromatography column included a dilute stream containing La and Ra that was diluted to 5 M HNO₃ prior to entry/loading of the dilute solution into the extraction chromatography column. In the example, the loading step utilized a 900 mL loading, requiring about a 1,500 mL total fluid volume. This large volume of dilute La/Ra solution was passed over an extraction chromatography column (a 10 mL volume column) that included BDGA resin. The La was able to be eluted from the column utilizing a volume of wash liquid of about 30 mL. In Example 1, one Cycle/Run was performed, which included three cycles/passes over the BDGA resin. Conditions for Example 1 are illustrated in Table 1 below.

TABLE 1
Conditions for Example 1

| [La] | 0.36 ppm in 5M HNO3 | |
|---|---|---|
| Starting Volume | 1 | L |
| Loading Flow Rate | 9 mL/min (60 rpm) | |
| BDGA BV | 10 | mL |
| Elution Fraction | 10 mL 0.05M HNO3 | |
| Elution Flow Rate | about 12 mL/min (80 rpm) | |
| # Cycles over BDGA | 3 | |
| Bottle Rinse Steps | 0 | |
| Total Time | 5 | hours |

| | |
|---|---|
| % La Recovered in 30 mL | 94% |

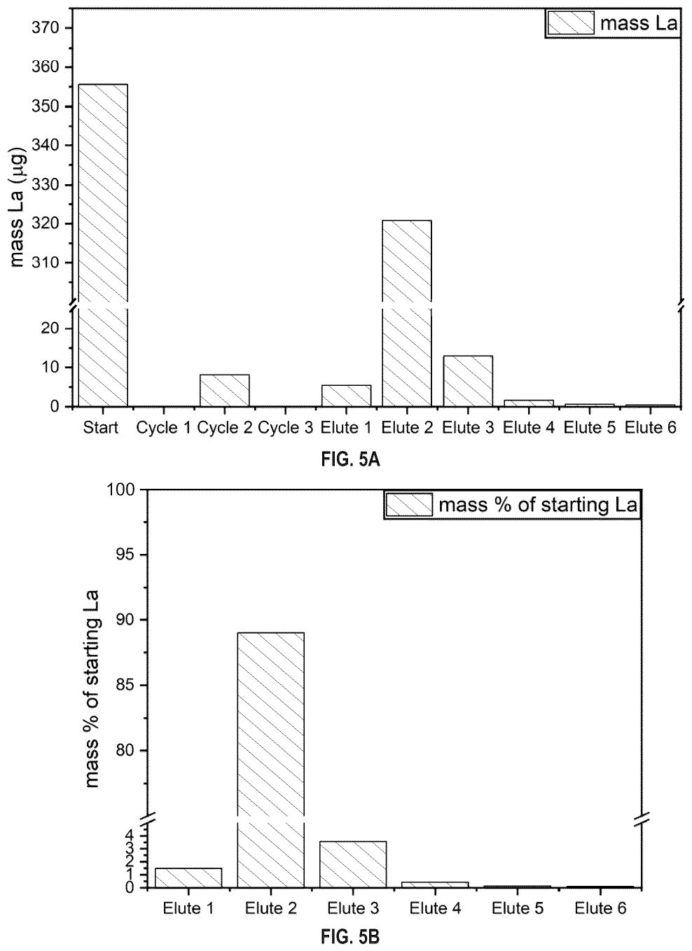

FIG. 5A illustrates experimental data showing a mass of La recovered during Example 1 over the course of the 3 cycles/passes and 6 elution fractions performed. FIG. 5B illustrates experimental data showing a mass percentage of La recovered during Example 1 over the course of the 6 elution fractions performed.

It was observed that it is possible that a single pass (cycle) over the BDGA resin nearly quantitatively retains the La.

### Example 2: La Over BDGA

Example 2 was performed utilizing the laboratory setup as described above with regards to Example 1. Again, La was utilized as an analog for Ac. In Example 2, two separate Experimental Cycles/Runs were performed, each only including one cycle/pass over the BDGA. Conditions for Example 2 are illustrated in Table 2 below.

TABLE 2
Conditions for Example 2

| Replicates | Cycle 1 | | Cycle 2 | |
|---|---|---|---|---|
| [La] | 0.987 ppm in 5M HNO3 | | 0.997 ppm in 5M HNO3 | |
| Starting Volume | 1 | L | 1 | L |
| Loading Flow Rate | 9 mL/min (60 rpm) | | 9 mL/min (60 rpm) | |
| BDGA BV | 10 | mL | 10 | mL |

| | | |
|---|---|---|
| Elution Fraction | 10 mL 0.05M HNO3 | 10 mL 0.05M HNO3 |
| Elution Flow Rate | about 12 mL/min (80 rpm) | about 12 mL/min (80 rpm) |
| # Cycles over BDGA | 1 | 1 |
| Bottle Rinse Steps | 0 | 0 |
| Total Time | about 2.3 hours | about 2.3 hours |
| % La Recovered in 30 mL | 95% | 95% |

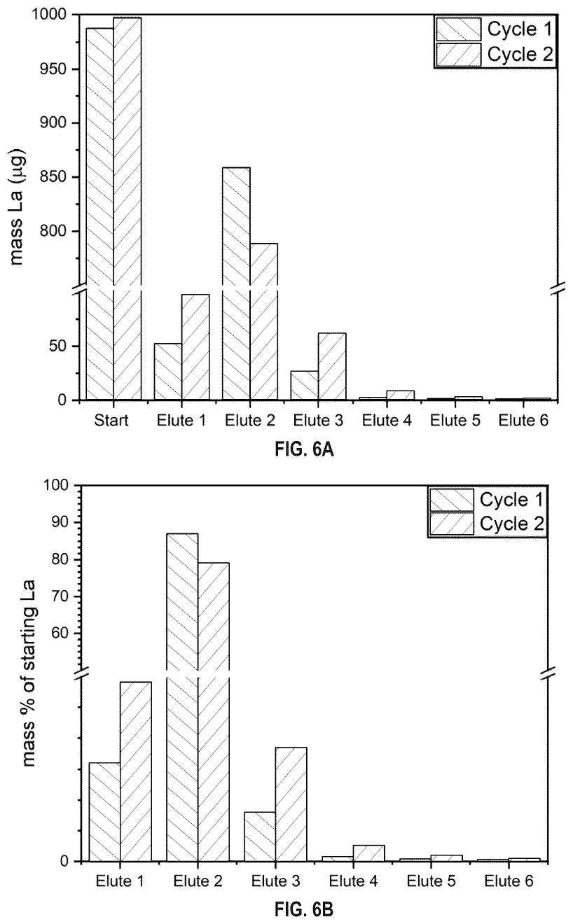

FIG. 6A illustrates experimental data showing a mass of La recovered during both single cycles of Example 2. FIG. 6B illustrates experimental data showing a mass percentage of La recovered during a both single cycles of Example 2. As seen in FIGS. 6A and 6B, Example 2 demonstrates reproducible distributions of eluted material.

It was observed that no La was detected in the 100× eluent passed through the column.

### Example 3: La and Th-232 Over BDGA

Example 3 was performed utilizing the laboratory setup as described above with regards to Example 1. Again, La was utilized as an analog for Ac. In Example 3, the starting solution included both La and Th-232 in a ratio of 1.55:1. The starting solution also included a less-characterized number of other impurities (for example, dissolved ThO₂O₂) to more closely mimic a composition of a solution that would be the outlet stream from a generator column as described herein. In Example 3, one Cycle/Run was performed, which included three cycles/passes over the BDGA resin. Conditions for Example 3 are illustrated in Table 3 below.

TABLE 3
Conditions for Example 3

| Ratio | La: 1.55 | Th232: 1 |
|---|---|---|
| [M] | 1.2 ppm in 5M HNO3 | 0.79 ppm in 5M HNO3 |
| Starting Volume | 1 L | |
| Loading Flow Rate | 9 mL/min (60 rpm) | |
| BDGA BV | 10 mL | |
| Elution Fraction | 10 mL 0.05M HNO3 | |
| Elution Flow Rate | about 12 mL/min (80 rpm) | |
| # Cycles over BDGA | 3 | |
| Bottle Rinse Steps | 0 | |
| Total Time | 5 hours | |
| % Mass Recovered in 30 mL | 80% | 15% |

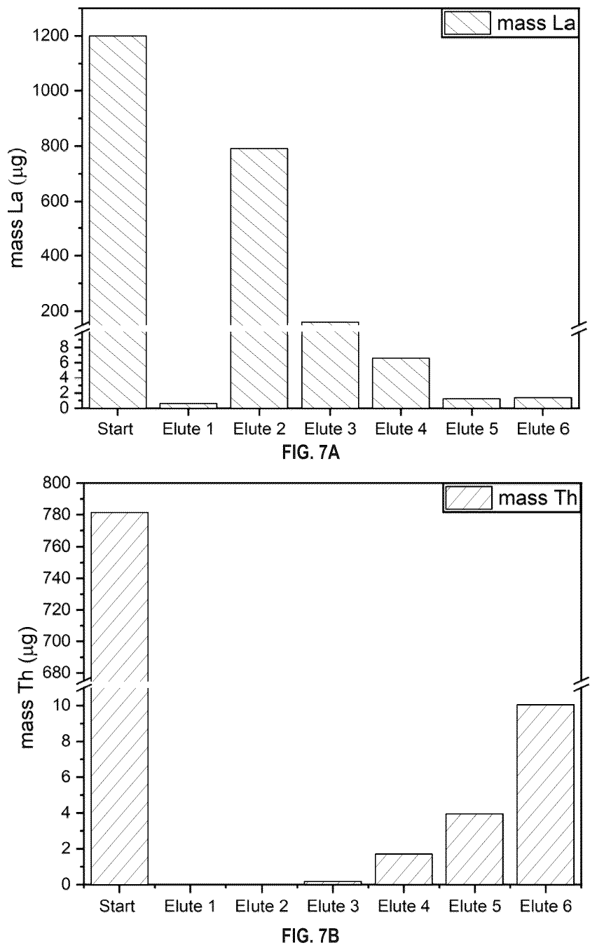

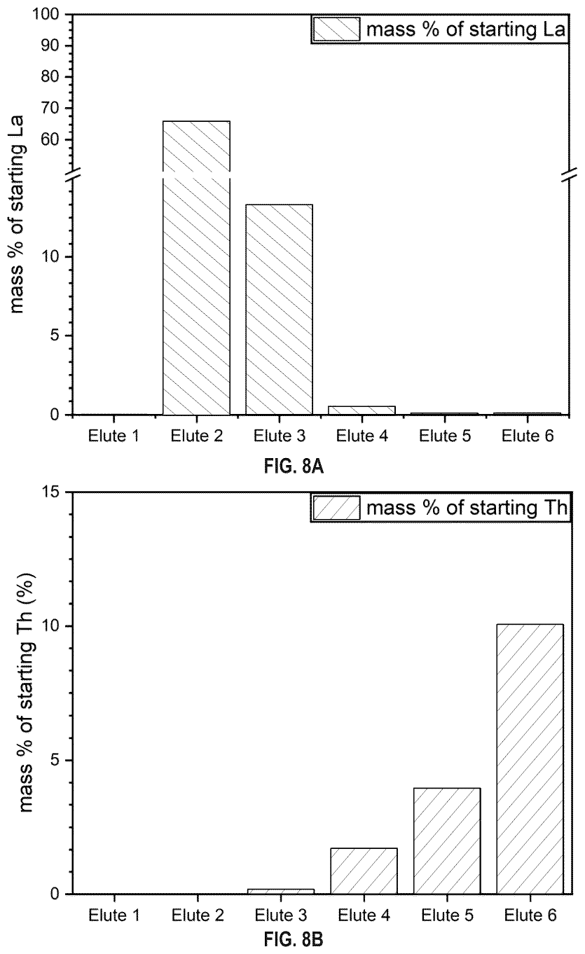

FIG. 7A illustrates experimental data showing a mass of La recovered during Example 3. FIG. 7B illustrates experimental data showing a mass of Th recovered during Example 3. FIG. 8A illustrates experimental data showing a mass percentage of La recovered during a Example 3. FIG. 8B illustrates experimental data showing a mass percentage of Th recovered during Example 3.

It was observed that there was a high retention of both La and Th after the first cycle/pass over the BDGA at 100× dilution. Lower overall recovery of La was seen in Example 3 than in examples 1 or 2, although about 80% of the mass of La was still recovered. It was also observed that the mass percentage of Th recovered was significantly lower than that of La, and more elution fractions were needed in order to achieve the observed recovery of Th.

Overall, the Examples 1, 2, and 3 illustrate that BDGA resins are suitable resins for concentration of La, even with surprisingly dilute solutions as a starting point. Based on the results and the characteristics of La and Ac, the conclusions are believed to be applicable to Ac.

### Example 4: Concentration of Ra

As described above, in some examples, a dilute Ra stream may be an output from a extraction chromatography column. The dilute Ra stream may be run through an additional column including an Ra-retaining resin to gather the Ra for further utilization. Table 4 below illustrates loading and elution conditions for the use of various potential resins for the concentration of the Ra from the dilute Ra stream. In examples, other resins may be utilized.

TABLE 4
Elution Conditions for Various Resins

| Material | Functional Group | Loading Condition | k′ | Elution Condition |
|---|---|---|---|---|
| 50Wx8 | Ar—SO3H | 1M HCl or <0.1M | about 1000 | 3-6M HNO3 |
| | | HNO3 | | |
| Sr Resin | 4,4′(5′)-di-t- | 1M HNO3 | 17 | 10M HNO3 |
| | butylcyclohexano | 12M HCl | 12 | |
| | 18-crown-6 | 0.5M HPF6 | 1200 | |
| | | 2.7M HClO4 | 1000 | |
| PbResin | 4,4′(5′)-di-t- | pH = 8, 10-7M LiCl, | 50000 | pH < 2 |
| | butylcyclohexano | Bengal Red | | |
| | 18-crown-6 | pH = 10, Cl— salt | 1200 | pH < 8 |
| LN Resin | HDHEP | 0.095M HNO3 | 1500 | 0.35M HNO3 |
| AnaLig Ra-01 | | 0.1-10M HCl or HNO3 | 12000 | pH = 10, 0.12M |
| | | | | NTA |
| TK 100 | 4,4′(5′)-di-t- | 0.01M HNO3 | 15000 about | 10M HNO3 |
| | butylcyclohexano | 1M HNO3 | 200 | |
| | 18-crown- | | | |
| | 6/HDHEP | | | |
| MnO2 | MnO2 | pH = 6-7 | | 4M HCl/1.5% |
| | | | | H2O2 |

It has been observed that, when utilizing HNOs, cation resins with higher crosslinking show better retention of Ra (Pt, and Au). Table 5 below illustrates expected process volume and dilution volume needed for the use of a particular cation resin (50W-X12 Resin) for the concentration of the Ra from the dilute Ra stream at different concentrations of HNOs.

TABLE 5
Projected Conditions for 50W-X12 Resin

| | Process volume | Dilution Volume | |
|---|---|---|---|
| M HNO3 | from BDGA | for 50wx12 | |
| 0.01 | 1.5 L | 750 | L |
| 0.1 | | 75 | L |
| 1 | | 7.5 | L |
| 2 | | 3.75 | L |
| 3 | | 2.25 | L |
| 4 | | 1.875 | L |

| | |
|---|---|
| 5 | No Dilution |

For a batch study utilizing 50W-X12 resin, freshly purified Ra-224/225 may be measured by gamma-spectroscopy. In a first step, the resin is equilibrated in the acid (HNO₃) for about 20 minutes. A spike of Ra in HNO₃ is then added to the system. The system is shaken for about one hour. The resulting mixture is filtered through an 0.45 μm filter. Samples for gamma-spectroscopy are then prepared, and finally counted.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained.

Notwithstanding that the numerical ranges and parameters setting forth the broad scope of the technology are approximations, the numerical values set forth in the specific examples are reported as precisely as possible. Any numerical value, however, inherently contain certain errors necessarily resulting from the standard deviation found in their respective testing measurements.

It will be clear that the systems and methods described herein are well adapted to attain the ends and advantages mentioned as well as those inherent therein. Those skilled in the art will recognize that the methods and systems within this specification may be implemented in many manners and as such are not to be limited by the foregoing exemplified embodiments and examples. In other words, functional elements being performed by a single or multiple components, e.g., columns, filters, or separators, could be combined or separated. In this regard, any number of the features of the different embodiments described herein may be combined into one single embodiment and alternate embodiments having fewer than or more than all of the features herein described are possible.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

While various embodiments have been described for purposes of this disclosure, various changes and modifications may be made which are well within the scope contemplated by the present disclosure. Numerous other changes may be made which will readily suggest themselves to those skilled in the art and which are encompassed in the spirit of the disclosure.

Although specific aspects were described herein, the scope of the technology is not limited to those specific aspects. One skilled in the art will recognize other aspects or improvements that are within the scope of the present technology. Therefore, the specific structure, acts, or operations are disclosed only as illustrative aspects. The scope of the technology is defined by the following claims and any equivalents therein. Therefore, the specific structure, acts, or operations are disclosed only as illustrative aspects. The scope of the technology is defined by the following claims and any equivalents therein. Examples of the disclosure may be described according to the following aspects.

Aspect 1. A method for concentrating Ac from a dilute stream containing Ac and Ra, the method comprising: providing the dilute stream containing Ac and Ra; passing the dilute stream through a column containing an extraction chromatography resin, thereby binding the Ac to the extraction chromatography resin and generating a dilute Ra stream; removing the dilute Ra stream from the column; after removing the dilute Ra stream from the column, washing the Ac from the extraction chromatography resin to generate a concentrated Ac stream; and removing the concentrated Ac stream from the column.

Aspect 2. The method of aspect 1, wherein the extraction chromatography resin is selected from N,N,N′,N′-tetra-n-octyldiglycolamide or N,N,N′,N′-tetra-2-ethylhexyldiglycolamide.

Aspect 3. The method of any of aspects 1-2, wherein the Ac is Ac-225.

Aspect 4. The method of any of aspects 1-3, wherein the Ra is Ra-225.

Aspect 5. The method of any of aspects 1-4, wherein the dilute stream containing Ac and Ra is generated from a column containing Th.

Aspect 6. The method of aspect 5, wherein the Th is Th-229.

Aspect 7. The method of any of aspects 1-6, further comprising passing the dilute Ra stream through a second column containing a second resin that binds the Ra to the second resin.

Aspect 8. The method of aspect 7, wherein the second resin is selected from among an Sr resin, a Pb resin, a cation resin, and an Ln resin.

Aspect 9. The method of any of aspects 1-8, wherein the dilute stream containing Ac and Ra comprises HNO3.

Aspect 10. The method of aspect 9, wherein the dilute stream containing Ac and Ra comprises HNO3 at a concentration of about 5 M HNO3.

Aspect 11. A method for concentrating Ac-225 from a dilute stream, the method comprising: preparing a column containing an extraction chromatography resin; contacting the dilute stream containing Ac-225 and Ra-225 with the extraction chromatography resin in the column, thereby loading at least a portion of the Ac-225 to the extraction chromatography resin; removing a dilute stream containing Ra-225 from the column; eluting the Ac-225-loaded extraction chromatography resin with a wash solution to produce an eluted solution; concentrating the eluted solution to generate eluted compounds; and separating the Ac-225 from other radioisotopes in the eluted compounds.

Aspect 12. The method of aspect 11, wherein the eluted solution comprises Ac-225 and comprises HNO3 at a concentration of about 0.05 M HNO3.

Aspect 13. The method of any of aspects 11-12, wherein the dilute stream comprises HNO3 at a concentration of about 5 M HNO3.

Aspect 14. The method of any of aspects 11-13, wherein the dilute stream is generated from a column containing Th and comprises at least one of HCl, NaNO3, HOA, NaOA, or HNO3.

Aspect 15. The method of any of aspects 11-14, wherein a column loading flow rate is about 9 mL/min.

Aspect 16. The method of any of aspects 11-15, wherein an elution speed of the wash solution is about 12 mL/min.

Aspect 17. The method of any of aspects 11-16, wherein the wash solution comprises HNO3.

Aspect 18. The method of aspect 17, wherein the wash solution comprises HNO3 in a range of 0.01-10M.

Aspect 19. A system, comprising: a first column; a first column body defining a first interior chamber; a first resin within the interior chamber, the first resin comprising an extraction chromatography resin capable of binding with Ac; a first selectable valve at an outlet of the first interior chamber, wherein a first position of the first selectable valve directs an outlet fluid containing eluted Ac from the first column to a second column; a second column body defining a second interior chamber; a second resin within the second interior chamber; and a second selectable valve at an outlet of the second interior chamber.

Aspect 20. The system of aspect 19, wherein the extraction chromatography resin is selected from N,N,N′,N′-tetra-n-octyldiglycolamide or N,N,N′,N′-tetra-2-ethylhexyldiglycolamide.

Aspect 21. The system of any of aspects 19-20, wherein the second resin comprises a UTEVA resin.

Aspect 22. The system of any of aspects 19-21, wherein an inlet to the first column receives a dilute stream comprising Ra and Ac from an upstream generator column containing Th.

Aspect 23. The system of aspect 22, wherein the dilute stream comprises HNO3 at a concentration of about 5 M HNO3.

Aspect 24. The system of any of aspects 19-23, wherein a position of the second selectable valve directs an Ra-containing outlet stream from the second column to a third column, the third column containing an Ra-retaining resin selected from among an Sr resin, a Pb resin, a cation resin, and an Ln resin.

Aspect 25. The system of any of aspects 19-24, wherein a position of the second selectable valve directs an Ac-containing outlet stream from the second column to an Ac-225 concentration process.

Aspect 26. A method of concentrating Ra from a dilute stream containing Ra and Ac, the method comprising: contacting the dilute stream with an extraction chromatography resin, generating a dilute Ra stream; contacting the dilute Ra stream with an Ra-retaining resin, thereby obtaining an Ra-loaded resin and a dilute waste stream; removing the dilute waste stream; after removing the dilute waste stream, passing a wash solution through a column containing the Ra-loaded resin, thereby removing at least some Ra from the Ra-loaded resin and generating a concentrated Ra stream; and removing the concentrated Ra stream.

Aspect 27. The method of aspect 26, wherein the extraction chromatography resin is selected from N,N,N′,N′-tetra-n-octyldiglycolamide or N,N,N′,N′-tetra-2-ethylhexyldiglycolamide.

Aspect 28. The method of any of aspects 26-27, wherein the Ac is Ac-225.

Aspect 29. The method of any of aspects 26-28, wherein the Ra is Ra-225.

Aspect 30. The method of any of aspects 26-29, wherein the dilute stream containing Ac and Ra is generated from a column containing Th.

Aspect 31. The method of aspect 30, wherein the Th is Th-229.

Aspect 32. The method of any of aspects 26-31, wherein the Ra-retaining resin is selected from among an Sr resin, a Pb resin, and an Ln resin.

Aspect 33. The method of any of aspects 26-32, wherein the Ra-retaining resin is a cation resin.

Aspect 34. The method of any of aspects 26-33, wherein the dilute stream containing Ac and Ra comprises HNO3.

Aspect 35. A method comprising: passing a dilute stream through a column containing an extraction chromatography resin, the dilute stream comprising a first component and HNO3 at a concentration of about 5 M HNO3, thereby loading at least a portion of the first component to the extraction chromatography resin to create a loaded resin and a column outlet stream; removing the column outlet stream from the column; eluting the loaded resin with a wash solution comprising an acid at a concentration of no greater than about 0.05 M, thereby releasing the first component from the resin and creating an eluent stream comprising the first component; and utilizing the first component of the eluent stream to generate a radiopharmaceutical.

Aspect 36. The method of aspect 35, wherein the eluent stream is not subject to an evaporation step prior to generation of the radiopharmaceutical.

Aspect 37. The method of any of aspects 35-36, wherein the first component comprises Ac.

Aspect 38. The method of aspect 37, wherein the first component comprises Ac-225.

Aspect 39. The method of any of aspects 35-38, wherein the acid comprises HNO3.

Aspect 40. The method of any of aspects 35-39, wherein the dilute stream comprises a second component, and wherein the column outlet stream comprises the second component.

Aspect 41. The method of aspect 40, wherein the second component comprises Ra.

Aspect 42. The method of aspect 41, wherein the second component comprises Ra-225.

Aspect 43. The method of any of aspects 35-42, wherein the extraction chromatography resin is selected from N,N,N′,N′-tetra-n-octyldiglycolamide or N,N,N′,N′-tetra-2-ethylhexyldiglycolamide.

What is claimed is:

1. A method for concentrating Ac from a dilute stream containing Ac and Ra, the method comprising:
providing the dilute stream containing Ac and Ra;
passing the dilute stream through a column containing an extraction chromatography resin, thereby binding the Ac to the extraction chromatography resin and generating a dilute Ra stream;
removing the dilute Ra stream from the column;
after removing the dilute Ra stream from the column, washing the Ac from the extraction chromatography resin to generate a concentrated Ac stream; and
removing the concentrated Ac stream from the column.

2. The method of claim 1, wherein the extraction chromatography resin is selected from N,N,N′,N′-tetra-n-octyldiglycolamide or N,N,N′,N′-tetra-2-ethylhexyldiglycolamide.

3. The method of claim 1, wherein the Ac is Ac-225.

4. The method of claim 1, wherein the Ra is Ra-225.

5. The method of claim 1, wherein the dilute stream containing Ac and Ra is generated from a column containing Th.

6. The method of claim 5, wherein the Th is Th-229.

7. The method of claim 1, further comprising passing the dilute Ra stream through a second column containing a second resin that binds the Ra to the second resin.

8. The method of claim 7, wherein the second resin is selected from among an Sr resin, a Pb resin, a cation resin, and an Ln resin.

9. The method of claim 1, wherein the dilute stream containing Ac and Ra comprises HNO₃.

10. The method of claim 9, wherein the dilute stream containing Ac and Ra comprises HNO₃ at a concentration of about 5 M HNO₃.

11. A method for concentrating Ac-225 from a dilute stream, the method comprising:
preparing a column containing an extraction chromatography resin;
contacting the dilute stream containing Ac-225 and Ra-225 with the extraction chromatography resin in the column, thereby loading at least a portion of the Ac-225 to the extraction chromatography resin;
removing a dilute stream containing Ra-225 from the column;
eluting the Ac-225-loaded extraction chromatography resin with a wash solution to produce an eluted solution;
concentrating the eluted solution to generate eluted compounds; and
separating the Ac-225 from other radioisotopes in the eluted compounds.

12. The method of claim 11, wherein the eluted solution comprises Ac-225 and comprises HNO₃ at a concentration of about 0.05 M HNO₃.

13. The method of claim 11, wherein the dilute stream comprises HNO₃ at a concentration of about 5 M HNO₃.

14. The method of claim 11, wherein the dilute stream is generated from a column containing Th and comprises at least one of HCl, NaNO₃, HOA, NaOA, or HNO₃.

15. The method of claim 11, wherein a column loading flow rate is about 9 mL/min.

16. The method of claim 11, wherein an elution speed of the wash solution is about 12 mL/min.

17. The method of claim 11, wherein the wash solution comprises HNO₃.

18. The method of claim 17, wherein the wash solution comprises HNO₃ in a range of 0.01-10M.

19. A system, comprising:
a first column;
a first column body defining a first interior chamber;
a first resin within the interior chamber, the first resin comprising an extraction chromatography resin capable of binding with Ac;
a first selectable valve at an outlet of the first interior chamber, wherein a first position of the first selectable valve directs an outlet fluid containing eluted Ac from the first column to a second column;
a second column body defining a second interior chamber;
a second resin within the second interior chamber; and
a second selectable valve at an outlet of the second interior chamber.

20. The system of claim 19, wherein the extraction chromatography resin is selected from N,N,N′,N′-tetra-n-octyldiglycolamide or N,N,N′,N′-tetra-2-ethylhexyldiglycolamide.

21. The system of claim 19, wherein the second resin comprises a UTEVA resin.

22. The system of claim 19, wherein an inlet to the first column receives a dilute stream comprising Ra and Ac from an upstream generator column containing Th.

23. The system of claim 22, wherein the dilute stream comprises HNO₃ at a concentration of about 5 M HNO₃.

24. The system of claim 19, wherein a position of the second selectable valve directs an Ra-containing outlet stream from the second column to a third column, the third column containing an Ra-retaining resin selected from among an Sr resin, a Pb resin, a cation resin, and an Ln resin.

25. The system of claim 19, wherein a position of the second selectable valve directs an Ac-containing outlet stream from the second column to an Ac-225 concentration process.

26. A method of concentrating Ra from a dilute stream containing Ra and Ac, the method comprising:
contacting the dilute stream with an extraction chromatography resin, generating a dilute Ra stream;
contacting the dilute Ra stream with an Ra-retaining resin, thereby obtaining an Ra-loaded resin and a dilute waste stream;
removing the dilute waste stream;
after removing the dilute waste stream, passing a wash solution through a column containing the Ra-loaded resin, thereby removing at least some Ra from the Ra-loaded resin and generating a concentrated Ra stream; and
removing the concentrated Ra stream.

27. The method of claim 26, wherein the extraction chromatography resin is selected from N,N,N′,N′-tetra-n-octyldiglycolamide or N,N,N′,N′-tetra-2-ethylhexyldiglycolamide.

28. The method of claim 26, wherein the Ac is Ac-225.

29. The method of claim 26, wherein the Ra is Ra-225.

30. The method of claim 26, wherein the dilute stream containing Ac and Ra is generated from a column containing Th.

31. The method of claim 30, wherein the Th is Th-229.

32. The method of claim 26, wherein the Ra-retaining resin is selected from among an Sr resin, a Pb resin, and an Ln resin.

33. The method of claim 26, wherein the Ra-retaining resin is a cation resin.

34. The method of claim 26, wherein the dilute stream containing Ac and Ra comprises HNO₃.

35. A method comprising:
passing a dilute stream through a column containing an extraction chromatography resin, the dilute stream comprising a first component and HNO₃ at a concentration of about 5 M HNO₃, thereby loading at least a portion of the first component to the extraction chromatography resin to create a loaded resin and a column outlet stream;
removing the column outlet stream from the column;
eluting the loaded resin with a wash solution comprising an acid at a concentration of no greater than about 0.05 M, thereby releasing the first component from the resin and creating an eluent stream comprising the first component; and
utilizing the first component of the eluent stream to generate a radiopharmaceutical.

36. The method of claim 35, wherein the eluent stream is not subject to an evaporation step prior to generation of the radiopharmaceutical.

37. The method of claim 35, wherein the first component comprises Ac.

38. The method of claim 37, wherein the first component comprises Ac-225.

39. The method of claim 35, wherein the acid comprises HNO₃.

40. The method of claim 35, wherein the dilute stream comprises a second component, and wherein the column outlet stream comprises the second component.

41. The method of claim 40, wherein the second component comprises Ra.

42. The method of claim 41, wherein the second component comprises Ra-225.

43. The method of claim 35, wherein the extraction chromatography resin is selected from N,N,N′,N′-tetra-n-octyldiglycolamide or N,N,N′,N′-tetra-2-ethylhexyldiglycolamide.
